(19) United States
(12) Patent Application Publication
(10) Pub. No.: US 2024/0033386 A1
(43) Pub. Date: Feb. 1, 2024

(54) A DISINFECTION SYSTEM, METHOD AND CHAMBER THEREOF

(71) Applicants: Vikas Khurana, BROOMALL, PA (US); Dinesh Kumar, New Delhi (IN)

(72) Inventors: Vikas Khurana, BROOMALL, PA (US); Dinesh Kumar, New Delhi (IN)

(21) Appl. No.: 18/020,423

(22) PCT Filed: Jul. 16, 2021
(86) PCT No.: PCT/IB2021/056453

§ 371 Date: Feb. 8, 2023

(30) Foreign Application Priority Data
Aug. 9, 2020 (IN) 202011015652

(51) Int. Cl. A61L 2/20; A61L 9/015

(52) U.S. Cl. CPC A61L 2/202; A61L 9/015; A61L 2209/212; A61L 2202/14; A61L 2202/25; A61L 2202/11; A61L 2209/111

(57) ABSTRACT

Disclosed is a disinfection arrangement (100) for disinfecting a chamber (132). The disinfection arrangement (100) includes an oxygen generation chamber (109) containing a mist generator (110) and a UV-C light source (118) positioned at an outlet of the oxygen generation chamber (109). The oxygen generation chamber (109) is configured to receive hydrogen-peroxide (H₂O₂) such that the received (H₂O₂) is exposed to the UV-C light source (118) at the outlet of the oxygen generation chamber (109) to convert the H₂O₂ into ozone for supplying the ozone to the chamber (132).

### FIELD OF INVENTION

The present embodiment relates to a disinfection system, and more particularly to ozone based disinfection chamber and system.

### BACKGROUND

The controlling of microbial contamination is one of the leading concerns in research, clinical, and medical facilities. Microorganisms (hazardous or not) can put personnel, patients and caregivers at risk. In hospital and medical facilities, patients are often compromised or have conditions that may make them particularly susceptible to opportunistic microbes or secondary infections.

Ultraviolet germicidal irradiation has been a mainstay for killing and inactivating microorganisms for well over a century. This process utilizes short-wavelength ultra-violet C light to kill microbes. UV-C light encompasses a range of 100 to 280 nm, but the most effective wavelength for decontamination is between 250-260 nm.

The exposure to the UV-C light inactivates microbial genomic DNA by creating lesions called thymine dimmers, which cannot be resolved by cellular-DNA repair mechanisms. This injury to cellular DNA impairs vital cellular functions and ultimately leads to the death of the microorganism. Thus, ultraviolet germicidal irradiation can be an effective means of sterilizing surfaces, instrumentation and facilities.

Hydrogen peroxide Vapor (HPV) is another effective way to sterilize and disinfect surfaces that are contaminated with undesirable microorganisms. The United States Environmental Protection Agency (EPA) classifies HPV as a disinfectant by virtue of its biocidal and pesticidal properties. The sterilization of surfaces and chambers with HPV involves release of a known concentration of vaporized hydrogen peroxide throughout an enclosed space, such as a lab or a room. The vapor is typically achieved with an HPV generator. These generators first remove moisture from ambient air and then pass liquid hydrogen peroxide past a vaporization module to produce a concentrated gaseous form of hydrogen peroxide.

The biocidal mechanism of action for hydrogen peroxide is attributed to chemical oxidation of cellular components. This oxidation rapidly interrupts vital chemical processes vital to microbial survival, and thus sterilizes the environment. For these reasons, HPV is used in many applications when personnel seek to disinfect environments contaminated by virulent microbes.

Of the two approaches, the UV-C decontamination approach is by far the most ubiquitous method for sterilization of closed spaces, surfaces and facilities. The rays can be easily generated with a short-wavelength light source. As such, these lights are mounted on surfaces or placed within chambers that require sterilization.

While UV-C irradiation is an inexpensive and effective form of microbial disinfection, care must be given to ensure proper personnel safety and the degree of disinfection required. HPV is arguably the most effective form of surface and facility decontamination, however quite hazardous to personnel. Therefore, it requires carefully calculating and documenting two important factors: the concentration of hydrogen peroxide used during cycles, and the time allowed for these vapours to disperse. An important advantage of HPV compared to UV-C sterilization is that line-of-sight is not a limiting factor; the gaseous biocide successfully decontaminates visible surfaces as well as hidden nooks and crannies. Additionally, hydrogen peroxide's biocidal properties have a much broader spectrum of targets. Bacterial spores, for example, are extremely susceptible to HPV decontamination. In fact, HPV sterilization is the method of choice by the US government in treating and decontaminating objects and structures after attacks using biological agents, such as Bacillus anthracis (anthrax). When comparing these two proven methods of decontamination, consider your applications, regulations and limitations. Both systems should be managed with care, as they present a potential risk to personnel safety. The decision to use one over the other requires you to look at the type of microorganisms common to your situation, time permitted to perform cleaning exercises, and budget. Such considerations may delay or at times jeopardize the process. While UV-C is cost effective and has less process time, it has limitations pertaining to less biocidal targets, reach of decontamination as well as being harmful to personnel whereas HPV with greater biocidal targets and reach for all surfaces is costly, time consuming as well as harmful to the personnel.

In view of the foregoing, there is a need for disinfecting systems and chambers that overcomes the limitations of both the established method and provides comprehensive benefits/advantages and there is a need to develop an air/oxygen blending system and related ventilation systems that is compact with low running and maintenance costs.

### SUMMARY OF THE PRIOR ART

A disinfecting arrangement includes a H₂O₂ supplier which is connected to a mist generator in a chamber via a H₂O₂ supply inlet. Furthermore, the chamber contains a H₂O₂ level sensor which is coupled to the mist generator.

Additionally, the disinfecting arrangement includes an UV-C light source.

The UV-C light source is connected to an air circulation system or a blower.

Furthermore, the ozone gas sensor and the humidity level sensor placed inside the chamber to monitor the level of ozone and humidity in the chamber.

Additionally, the air circulation system is coupled with an air suction fan.

The disinfecting arrangement further includes an UV-B light source which is connected to the chamber. The UV-B light source is further connected to the gas exit valve (160).

### BRIEF DESCRIPTION OF DRAWINGS

The drawing/s mentioned herein discloses exemplary embodiments of the claimed invention. Detailed description and preparation of well-known compounds/substances/elements are omitted to not unnecessarily obscure the embodiments herein. Other objects, features, and advantages of the present invention will be apparent from the following description when read with reference to the accompanying drawing.

FIG. 1 illustrates a disinfecting arrangement (100), according to an embodiment herein.

FIG. 2 illustrates the perspective view of a hyperbaric chamber (200), according to an embodiment herein and a disinfection apparatus (200) according to an aspect herein; and

FIG. 3 illustrates a flowchart of disinfection method (400), according to an embodiment herein.

To facilitate understanding, like reference numerals have been used, where possible to designate like elements common to the figures.

### DETAILED DESCRIPTION

This section is intended to provide explanation and description of various possible embodiments of the present invention. The embodiments used herein, and the various features and advantageous details thereof are explained more fully with reference to non-limiting embodiments illustrated in the accompanying drawing/s and detailed in the following description. The examples used herein are intended only to facilitate understanding of ways in which the embodiments may be practiced and to enable the person skilled in the art to practice the embodiments used herein. Also, the examples/embodiments described herein should not be construed as limiting the scope of the embodiments herein.

In an embodiment, the word chamber and hyperbaric chamber are used interchangeable in the context.

In an embodiment, atomizer and mist generator are used interchangeable in the context.

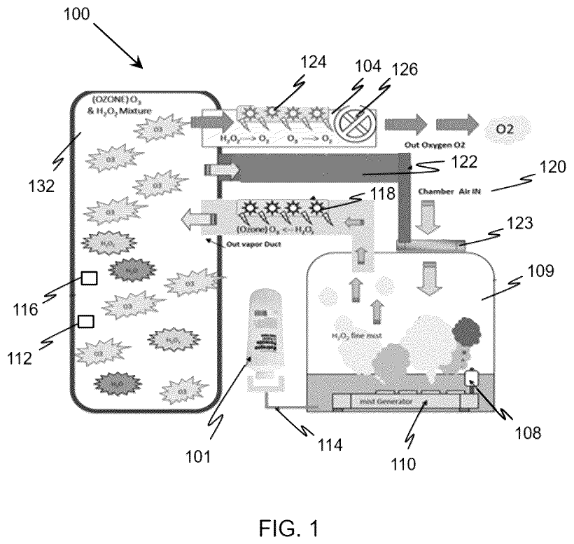

Referring to FIG. 1, a disinfecting arrangement (100) for disinfection of a chamber (102) is disclosed.

The disinfecting arrangement (100) includes a chamber (102), an oxygen generation chamber (104), a H₂O₂ supplier (106), a H₂O₂ level sensor (108), a H₂O₂ supply inlet (110), an atomizer or mist generator (112), a humidity sensor (114), an ozone gas sensor (116), an ultraviolet-C(UV-C) light source (118), an air circulation system (120), an air suction fan (122), an ultraviolet-B (UV-B) light source (124) and a gas exit valve (126).

The H₂O₂ supplier (106) is connected to the mist generator (112) in the oxygen generation chamber (104) via the H₂O₂ supply inlet (110). The oxygen generation chamber (104) contains the H₂O₂ level sensor (108), which is coupled to the mist generator (112). The chamber (102) contains the humidity sensor (114) and the ozone gas sensor (116).

The UV-C light source (120) is disposed at the outlet of the oxygen generation chamber (104) of the disinfecting arrangement (200). The chamber (102) and the oxygen generation chamber (104) are operatively coupled through the air circulation system (120). The air circulation system (120) is configured to throw the ozone from the chamber (102) to the oxygen generation chamber (104) by virtue of the air suction fan (122) of the air circulation system (120).

The UV-B light source (124) is disposed at the outlet of the chamber (102). The gas exit valve is (126) is provided downstream the UV-B light source (124).

In an embodiment, the H₂O₂ supplier (106) may be a tank, a motor or a pump.

In an embodiment, the atomizer (112) may be a fog generator, a fogger, a mist maker, an ultrasonic mist maker/generator, a piezo atomizer or an ultrasonic atomizer.

In another embodiment, the H₂O₂ level sensor (108) may include a gas sensor such as MQ2 gas sensor, a grove-gas sensor, fuel level sensor and a mechanical resistive-based sensor.

In an embodiment, the H₂O₂ supply inlet (110) may be a simple hose pipe.

In an embodiment, the UV-C light source (118) may be a germicidal lamp, germicidal bulb, an UV-C light torch, lamp, thrower etc.

In an embodiment, the chamber (102) is any enclosed room, a hyperbaric chamber, a capsule-type-room.

In an embodiment, the air circulation system (120) may be a ventilation system, an airing system.

In an embodiment, the UV-B light source (124) may be a lamp, a UV-B emitting LEDs, a UV-B bulb, torch, lamp, thrower etc.

In an embodiment, the gas exit valve (126) may be an exhaust system.

The H₂O₂ supplier (106) is configured to supply H₂O₂ composition to the mist generator (112). It is well known in the art that hydrogen peroxide (H₂O₂) is an oxidizing agent and used as an oxidizer, bleaching agent, and an antiseptic. Due to its high oxidation potential and strong performance across a wide pH range it is extensively used in industries as a biocide.

Hydrogen per oxide exhibits broad-spectrum activity including its efficacy against bacterial endospores that leads to degradation of bacterial growth. H₂O₂ is particularly interesting for its application in liquid but also vaporized form for antisepsis and for the disinfection of surfaces and medical devices and for room fumigation.

The H₂O₂ supplier (106) supplies H₂O₂ compound to the mist generator (112) via the H₂O₂ supply inlet (110) in a required concentration. The mist generator (112) has a mechanism to generate or form fine H₂O₂ mist or cloud in the oxygen generation chamber (104). Fine H₂O₂ mist provides rich source of oxygen. Higher concentration of Ozone may be achieved compared to room air oxygen using ultrasonic atomizer crystal to develop fine mist. This enables instantaneous and harmless disinfection of the room at lower cost. The H₂O₂ level sensor (108) placed inside the oxygen generation chamber (104) monitors the level/concentration of the H₂O₂ input and the mist output. In a preferred embodiment, the atomizer (112) has a capacity of generating 1.5 litre/hours of 12% H₂O₂.

The humidity sensor (114) placed in the chamber (102) measures the level of humidity which is H₂O (water) so that it does not cross a certain level and maintains the idle environment for disinfection in the chamber.

The ozone gas sensor (116) placed inside the chamber (102) measures the level of ozone gas supplied to the chamber (102) so that it does not cross a certain level and maintains the idle gas environment for disinfection in the chamber (130).

The UV-C light source (118) disposed at the outlet of the oxygen generation chamber (104) is configured to convert the H₂O₂ mist to the ozone molecules via oxidation process/chemical reaction. The UV-C light encompasses a range of 100 to 280 nm, but the most effective wavelength for decontamination is between 250-260 nm.

The general process according to an embodiment herein includes breaking H₂O₂ into water and oxygen using catalase, an enzyme that is found in microbes, and then switching on UV-C light source that makes ozone from the released oxygen (from H₂O₂).

This process utilizes short-wavelength ultraviolet-C light to kill microbes. UV-C light encompasses a range of 100 to 280 nm, but the most effective wavelength for decontamination is between 250-260 nm. UV germicidal irradiation is only effective in sterilizing surfaces that are in the ray's line of sight. Any portion of a surface that is “hidden” by other objects will not be exposed, and therefore cannot be sterilized. Operator safety is another obvious concern as UV-C light damages microbial DNA, irradiation can also cause DNA damage in humans. Thus, procedures need to include instructions for keeping personnel at a safe distance from these light sources. Thus, care must be given to ensure proper personnel safety and the degree of disinfection required. It has been shown that humidity increases the efficiency of ozone. Therefore, a humidity level senor (114) is placed inside the chamber (102) to maintain the level of humidity.

The mist generator (112) produces 5 μm H₂O₂ droplets. The mist generator uses atomizing crystal to form particles with size <10 um to 200 um using 1.7 Mhz.

In a preferred embodiment, the mist has 12% H₂O₂. The housing includes the air circulation system (120) so as to expose H₂O₂ mist to catalase in the microbes [in the air] to break the hydrogen peroxide to release oxygen and water. The oxygen and H₂O₂ mixture is then passed through the chamber (102) or enclosure fitted with UV-C light source (118) to convert the released Oxygen to Ozone, which is then directed to different applications and rooms/chambers (102) such as for disinfecting a wardrobe containing apparel of hospital staff or an enclosed room.

The UV-B light source (124) is provided at the outlet of the chamber (124) or wardrobe or an enclosed room for converting ozone to oxygen by UV-B light exposure for destruction of ozone from exhaust for environment protection by UV-B. The arrangement also includes a control panel for controlling/monitoring duration and safety. The gas exit valve (126) enables the controlled rate of flow of oxygen outside to the environment. Furthermore, the moisture and oxygen may also be ejected out from the gas exit valve (126) to the environment (internal or external).

The regulatory path for decontaminating spaces is guided by CDC, OSHA, FDA and Environmental Protection Agency. Disinfection of patient areas has been on the forefront for CDC and FDA however limited alternatives exist. Environment friendly ozone disinfection process can be used for disinfecting patient beds in the hospital, airplanes, operating theaters, and various other enclosed spaces. For early commercialization, the disinfection arrangement (200) may be used to disinfect reusable medical devices as approved by FDA (880.6890—General purpose disinfectants).

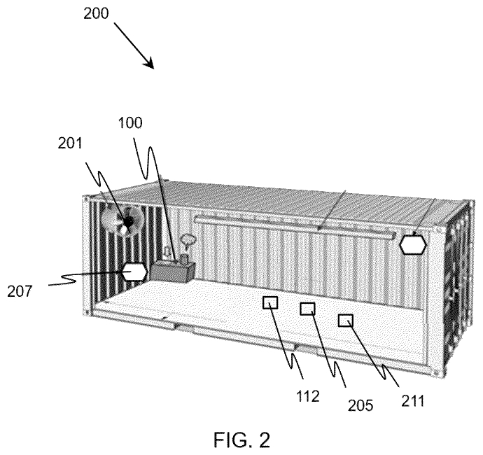

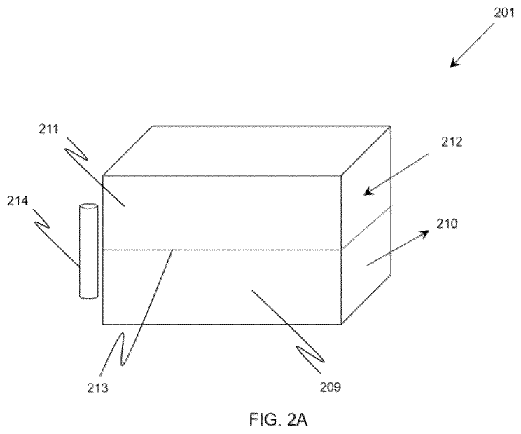

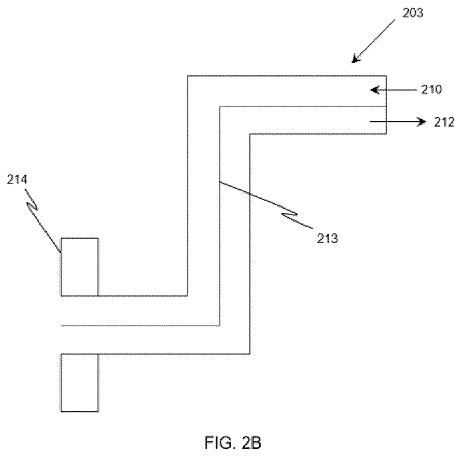

Referring to FIG. 2, a hyperbaric chamber (200) in accordance with an embodiment of the present disclosure is disclosed.

The hyperbaric chamber (200) includes the H₂O₂ supply inlet (110), the mist generator (112), the air circulation system (120), the humidity level sensor (114), the ozone gas sensor (116) and the UV-C light source (118).

The hyperbaric chamber (200) includes the supply inlet (110), which is connected to the mist generator (112). The chamber (200) includes the UV-C light source (118) which is positioned inside the chamber (200). The chamber (200) is equipped with the air circulation system (120). The chamber (200) includes the humidity level sensor (114) and ozone gas sensor (116).

As used herein, the hyperbaric chamber (200) includes the supply inlet (110), which takes H₂O₂ compound from outside located H₂O₂ supplier (106). The supply inlet (110) transmits the H₂O₂ compound to the mist generator (112). The mist generator (112) produces H₂O₂ mist to the chamber (200). The mist generator (112) has a mechanism to generate or form fine H₂O₂ mist or cloud to the chamber (200).

The UV-C light source (118) located in the chamber (200) converts the H₂O₂ mist to the ozone and water (humidity) molecules via oxidation process/chemical reaction.

The emitted water, if not measured correctly may lead to decontamination of the chamber (200). Therefore, the humidity level sensor (114) is placed inside the chamber (200) to measure the level of humidity in the chamber (200).

The ozone gas sensor (116) placed inside the chamber (200) measures the level of ozone gas supplied to the chamber (200) so that it does not cross a certain level and maintains the idle gas environment for disinfection in the chamber (200).

In a preferred embodiment, the mist has 12% H₂O₂. The housing includes the air circulation system (120) so as to expose H₂O₂ mist to catalase in the microbes [in the air] to break the hydrogen peroxide to release oxygen and water. The oxygen and H₂O₂ mixture is then passed through the chamber (200) or enclosure fitted with UV-C light source (118) to convert the released Oxygen to Ozone, which is then directed to different applications and rooms/chambers (200) such as for disinfecting a wardrobe containing apparel of hospital staff or an enclosed room.

After the disinfection process, ozone is ejected out from the O₃ exit valve located inside the chamber, where the ozone is converted to oxygen by the UV-B light source located at the outlet of the chamber (200).

In an aspect, a disinfection apparatus (200) for disinfecting a region (204) includes an oxygen generation chamber (128) containing a mist generator (110) and a UV-C light source (120) positioned at an outlet of the oxygen generation chamber (128).

The oxygen generation chamber (128) is configured to receive hydrogen-peroxide (H₂O₂) to convert (H₂O₂) into oxygen and water inside the oxygen generation chamber (128). The generated oxygen is exposed to the UV-C light source (120) at the outlet of the oxygen generation chamber (128) to convert the oxygen into ozone for supplying the ozone into the region (204) of the disinfection apparatus (200).

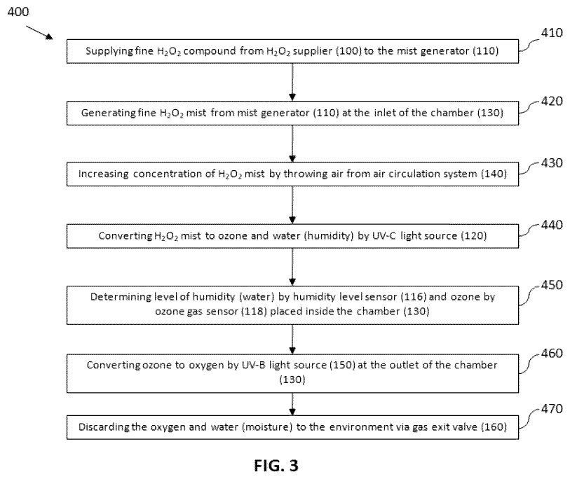

Referring to FIG. 3, a flowchart of method of disinfection (400) of hyperbaric chamber (200), in accordance with an embodiment of the present disclosure is disclosed.

At step 410, the H₂O₂ supplier (106) supplies H₂O₂ composition to the mist generator (112).

At step 420, the mist generator (112) generates H₂O₂ mist to the inlet of the chamber (200).

At step 430, the air circulation system (120) blows ozone to the chamber (200) to increase concentration of H₂O₂ mist inside the chamber (200).

At step 440, the UV-C light source (118), which is located inside the chamber (200), converts the H₂O₂ to the ozone and water (humidity) inside the chamber (200).

At step 450, the humidity level sensor (114) monitors the level of the humidity in the chamber (200) to maintain the required level of humidity and the ozone gas sensor (116) monitors the level of the ozone gas quantity in the chamber (200).

At step 460, the UV-B light source (124), which is located at the outlet of the chamber (200) converts ozone to the oxygen after disinfection of the chamber (200).

At step 470, the gas exit valve (126) ejects the oxygen to the environment.

The emitted oxygen may be used for many activities including but not limited to store it for medical purposes, to use it for machinery purposes, to use it by the petroleum industry.

In an aspect, a hyperbaric chamber (200) with an air flow arrangement (207) for disinfecting a region (204) is provided. The hyperbaric chamber (200) with an airflow arrangement has an air flow arrangement that is configured to facilitate heat exchange between the region (204) and the surroundings of the hyperbaric chamber (200). The air flow arrangement (207) further includes at least a pair of air circulating device (209) one of which is configured to suck in the air into the region (204) and the other is configured to blow out the air from the region (209). The air circulating device (209) is a linear fan adapted for maximizing the area of heat exchange between the hyperbaric chamber (200) and the surroundings of the hyperbaric chamber (200). The air flow arrangement incorporates a heat exchanging foil such as an aluminum foil for exchanging heat between the region (204) and surroundings of the hyperbaric chamber (200). The hyperbaric chamber is provided with a plurality of volatile organic compound (VOC) sensors that are configured to monitor VOC level inside the region (204) of the hyperbaric chamber (200). An ozone trigger (211) is configured to release ozone when VOC value exceeds a threshold value inside the region (204) of the hyperbaric chamber (200). The air flow arrangement is powered by a solar panel. In an embodiment, the aluminum foil is replaceable and the aluminum foil is supported by shock absorbing mechanism.

We claim:

1. A disinfection arrangement (100) for disinfecting a chamber (130) comprising;
an oxygen generation chamber (104) containing a mist generator (112) and a UV-C light source (120) positioned at an outlet of the oxygen generation chamber (104);
the oxygen generation chamber (104) is configured to receive hydrogen-peroxide (H₂O₂) to convert (H₂O₂) into oxygen and water inside the oxygen generation chamber (104); wherein
the generated oxygen is exposed to the UV-C light source (120) at the outlet of the oxygen generation chamber (104) to convert the oxygen into ozone for supplying the ozone to the chamber (102).

2. The disinfection arrangement (100) as claimed in claim 1, wherein a humidity sensor (116) is placed inside the hyperbaric chamber (102) to measure humidity level so produced during conversion of (H₂O₂) into oxygen and water inside the oxygen generation chamber (104).

3. The disinfection arrangement (100) as claimed in claim 1, wherein an ozone gas sensor (118) is placed inside the hyperbaric chamber (102) to measure quantity of ozone gas level inside the hyperbaric chamber (102).

4. The disinfection arrangement (100) as claimed in claim 1, wherein a UV-B light source (124) is disposed at the outlet of the hyperbaric chamber (102) to convert the ozone back to the oxygen.

5. The disinfection arrangement (100) as claimed in claim 1, wherein the UV-B light source (124) is further connected to a gas exit valve (160) to throw out oxygen to the environment.

6. The disinfection arrangement (100) as claimed in claim 1, wherein a H₂O₂ supplier (100) is connected to the mist generator (110) to supply H₂O₂ compound.

7. The disinfection arrangement (100) as claimed in claim 1, wherein a (H₂O₂) level sensor (112) is placed inside the oxygen generation chamber (104) to measure fine mist of (H₂O₂) produced inside the oxygen generation chamber (104).

8. The disinfection arrangement (100) as claimed in claim 1, wherein an air circulation system (140) is operatively coupled between the chamber (102) and the oxygen generation chamber (104) to expose H₂O₂ mist to catalase in the microbes to break the hydrogen peroxide to release oxygen and water.

9. The disinfection arrangement (100) as claimed in claim 1, wherein the air circulation system (140) further comprising an air suction fan (142) configured to throw ozone to the oxygen generation chamber (104).

10. A hyperbaric chamber (200) comprising;
a UV-C light source (120) positioned at the inlet of the hyperbaric chamber (200) and a UV-B light source (124) positioned at the outlet of the hyperbaric chamber (200);
the UV-C light source (120) is configured to convert oxygen into ozone that is supplied into the hyperbaric chamber (200) for disinfecting the hyperbaric chamber (200) and the UV-B light source (124) is configured to convert ozone back into oxygen; wherein
a humidity sensor (116) is placed inside the hyperbaric chamber (200) to measure humidity level inside the hyperbaric chamber (102).

11. The hyperbaric chamber (200) as claimed in claim 10, wherein an oxygen generation chamber (104) is coupled to the hyperbaric chamber (200) that is configured to generate oxygen and water.

12. The hyperbaric chamber (200) as claimed in claim 10, wherein the UV-B light source (124) is further connected to a gas exit valve (160) to throw out oxygen to the environment.

13. The hyperbaric chamber (102) as claimed in claim 10, wherein an air circulation system (140) is positioned inside the hyperbaric chamber (102) to expose H₂O₂ mist to catalase the microbes to break the hydrogen peroxide to release oxygen and water.

14. The hyperbaric chamber (102) as claimed in claim 10, wherein the air circulation system (140) further comprising an air suction fan (142) configured to throw ozone to the oxygen generation chamber (104).

15. A disinfection apparatus (200) for disinfecting a region (204) comprising;
an oxygen generation chamber (104) containing a mist generator (110) and a UV-C light source (120) positioned at an outlet of the oxygen generation chamber (104);
the oxygen generation chamber (104) is configured to receive hydrogen-peroxide (H₂O₂) to convert (H₂O₂) into oxygen and water inside the oxygen generation chamber (104); wherein
the generated oxygen is exposed to the UV-C light source (120) at the outlet of the oxygen generation chamber (104) to convert the oxygen into ozone for supplying the ozone into the region (204) of the disinfection apparatus (200).

16. The disinfection apparatus (200) as claimed in claim 15, wherein the ozone is exposed to UV-B light source at the outlet of the region (204) for converting ozone back into oxygen.

17. The disinfection apparatus (200) as claimed in claim 15, wherein a humidity sensor (116) is placed inside the region (204) to measure humidity level so produced during conversion of (H₂O₂) into oxygen and water inside the oxygen generation chamber (104).

18. The disinfection apparatus as claimed in claim 15, wherein an air circulation system (140) is positioned inside the region (204) to throw ozone to the oxygen generation chamber (104).

19. A method of disinfecting a hyperbaric chamber (200) comprising;
receiving hydrogen-per-oxide (H₂O₂) inside an oxygen generation chamber (104);
generating oxygen by an oxygen generation chamber (104) containing a mist generator (110) configured to produce oxygen and water from hydrogen per oxide (H₂O₂); wherein
exposing the generated oxygen to the UV-C light source (120) at the outlet of the oxygen generation chamber (104) to convert the oxygen into ozone;
supplying the ozone to the hyperbaric chamber (200).

20. The method of disinfecting the hyperbaric chamber (200) as claimed in claim 19, wherein the ozone is exposed to UV-B light source at the outlet of the hyperbaric chamber (200) for converting ozone back into oxygen.

21. The method of disinfecting the hyperbaric chamber (200) as claimed in claim 19, wherein a humidity sensor (116) is placed inside the hyperbaric chamber (200) to measure humidity level so produced during conversion of (H₂O₂) into oxygen and water inside the oxygen generation chamber (104).

22. The method of disinfecting the hyperbaric chamber (200) as claimed in claim 19, wherein an air circulation system (140) is positioned inside the hyperbaric chamber (102) to expose H₂O₂ mist to catalase the microbes to break the hydrogen peroxide to release oxygen and water.

23. A hyperbaric chamber (200) with a dehumidifier (203) comprising;
a UV-C light source (120) positioned at the inlet of the hyperbaric chamber (200) and a UV-B light source (124) positioned at the outlet of the hyperbaric chamber (200);
the UV-C light source (120) is configured to convert oxygen into ozone that is supplied into the hyperbaric chamber (200) for disinfecting the hyperbaric chamber (200); wherein
the dehumidifier (203) is disposed inside the hyperbaric chamber (200) configured to dehumidify the hyperbaric chamber (200).

24. The hyperbaric chamber (200) as claimed in claim 23, wherein the dehumidifier dehumidifies the chamber (200) upon measuring the humidity level by a humidity sensor (116) placed inside the hyperbaric chamber (102).

25. The hyperbaric chamber (200) as claimed in claim 23, wherein the chamber (200) further comprising a plurality of volatile organic compound (VOC) sensors that are configured to monitor VOC level inside the region (204) of the hyperbaric chamber (200).

26. The hyperbaric chamber (200) as claimed in claim 23, wherein, an ozone trigger (211) is configured to release ozone when VOC value exceeds a threshold value inside the region (204) of the hyperbaric chamber (200).

27. A hyperbaric chamber (200) with an air flow arrangement (207) for disinfecting a region (204) comprising;
a UV-C light source (120) positioned at the inlet of the hyperbaric chamber (200) and a UV-B light source (124) positioned at the outlet of the hyperbaric chamber (200);
the UV-C light source (120) is configured to convert oxygen into ozone that is supplied into the hyperbaric chamber (200) for disinfecting the region (204) of the hyperbaric chamber (200); wherein
the air flow arrangement is configured to facilitate heat exchange between the region (204) and the surroundings of the hyperbaric chamber (200).

28. The hyperbaric chamber (200) as claimed in claim 27, wherein the air flow arrangement (207) further comprising at least a pair of air circulating device (209) one of which is configured to suck in the air into the region (204) and the other is configured to blow out the air from the region (209).

29. The hyperbaric chamber (200) as claimed in claim 27, wherein the air circulating device (209) is a linear fan adapted for maximizing the area of heat exchange between the hyperbaric chamber (200) and the surroundings of the hyperbaric chamber (200).

30. The hyperbaric chamber (200) as claimed in claim 27, wherein the air flow arrangement incorporates a heat exchanging foil such as an aluminum foil for exchanging heat between the region (204) and surroundings of the hyperbaric chamber (200).

31. The hyperbaric chamber (200) as claimed in claim 27, wherein the hyperbaric chamber is provided with a plurality of volatile organic compound (VOC) sensors that are configured to monitor VOC level inside the region (204) of the hyperbaric chamber (200).

32. The hyperbaric chamber (200) as claimed in claim 27, wherein an ozone trigger (211) is configured to release ozone when VOC value exceeds a threshold value inside the region (204) of the hyperbaric chamber (200).

33. The hyperbaric chamber (200) as claimed in claim 27, wherein the air flow arrangement is powered by a solar panel.

34. The hyperbaric chamber (200) as claimed in claim 30, wherein the aluminum foil is replaceable.

35. The hyperbaric chamber (200) as claimed in claim 30, wherein the aluminum foil is supported by shock absorbing mechanism.
